(19) United States
(12) Patent Application Publication
(10) Pub. No.: US 2009/0318502 A1
(43) Pub. Date: Dec. 24, 2009

(54) Pharmaceutical Compositions Comprising a Hypoglycemic Agent and Methods of Using Same

(75) Inventors: Greg Plucinski, Nicholasville, KY (US); Mehdi Rezaian-Yazdi, Columbia, SC (US)

Correspondence Address: GOODWIN PROCTER LLP;PATENT ADMINISTRATOR, 53 STATE STREET, EXCHANGE PLACE, BOSTON, MA, 02109-2881, US

(73) Assignee: INTRANASAL THERAPEUTICS, INC., Lexington, KY (US)

(21) Appl. No.: 12/293,843

(22) PCT Filed: Mar. 22, 2007
(86) PCT No.: PCT/US2007/007102

§ 371 Date: Mar. 10, 2009

Related U.S. Application Data
(60) Provisional application No. 60/784,946, filed on Mar. 22, 2006.

(51) Int. Cl. A61K 31/4453; A61P 3/10

(52) U.S. Cl. 514/331

(57) ABSTRACT

The present invention relates to intranasally deliverable compositions comprising a hypoglycemic agent, for example, repaglinide, and to methods of using such compositions in the treatment of various disorders, including, for example, type-2 diabetes.

### RELATED APPLICATIONS

This application claims the benefit of priority to U.S. Provisional Patent Application Ser. No. 60/784,946, filed Mar. 22, 2006, the contents of which are hereby incorporated by reference.

### FIELD OF THE INVENTION

The present invention relates to pharmaceutical compositions comprising a hypoglycemic agent and to methods of using such compositions to treat and/or prevent various diseases and disorders.

### BACKGROUND

Subjects with insulin related disorders such as type-2 diabetes commonly exhibit post-meal (postprandial) spikes in blood-sugar levels. If untreated, such postprandial blood sugar increases can cause long-term damage to the heart and other organs.

Repaglinide (Prandin®) is currently marketed as an oral tablet for treating postprandial increases in blood sugar levels. Repaglinide is believed to act by stimulating beta cells in the pancreas to produce insulin. Unfortunately, repaglinide must be administered within a fairly precise window of 15-30 minutes before eating, otherwise the therapeutic benefit of repaglinide can be significantly diminished.

There is still an ongoing need for formulations containing a hypglycemic agent, for example, repaglinide, that can be delivered quickly and that maintain the therapeutic benefit of the active ingredient.

### SUMMARY OF THE INVENTION

In one aspect, the present invention provides intranasally deliverable pharmaceutical compositions comprising a hypoglycemic agent and methods of using such compositions for treating and/or preventing various diseases and disorders, for example, type-2 diabetes. In one embodiment, the hypoglycemic agent is a nonsulfonylurea hypoglycemic agent. In another embodiment, the hypoglycemic agent is a meglitinide. In still another embodiment, the hypoglycemic agent is a phenylacetic acid benzylamide.

In one embodiment, the composition comprises a compound of Formula I:

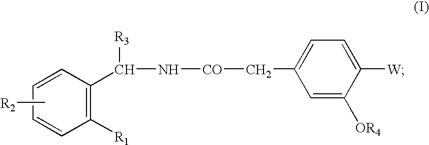

including salts, esters, and prodrugs thereof, wherein R₁, R₂, R₃, R₄ and W are described in more detail herein below.

In another embodiment, the composition comprises a compound of Formula II:

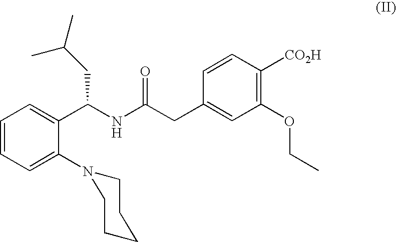

including salts, esters, and prodrugs thereof.

In another aspect, the invention provides an intranasal formulation comprising:
(a) from 0.5% to 5% (w/v) repaglinide;
(b) from 0% to 10% (v/v) ethanol;
(c) from 0% to 10% (v/v) propylene glycol;
(d) from 30% to 60% (v/v) polyethylene glycol (PEG), for example, PEG-200, PEG-300, or PEG-400;
(e) from 0% to 20% (v/v) methoxy-polyethylene glycol;
(f) from 0% to 40% (v/v) tetra(ethylene glycol);
(g) from 0% to 10% v/v phosphate buffer; and
(h) less than 30% (v/v) of other excipients.

In another embodiment, the invention provides an intranasal formulation comprising:
(a) from 1% to 2% (w/v) repaglinide;
(b) from 0% to 10% (v/v) ethanol;
(c) from 0% to 10% (v/v) propylene glycol;
(d) from 50% to 60% (v/v) polyethylene glycol (PEG), for example, PEG-200, PEG-300, or PEG-400;
(e) from 0% to 20% (v/v) methoxy-polyethylene glycol;
(f) from 0% to 40% (v/v) tetra(ethylene glycol);
(g) from 0% to 10% v/v phosphate buffer; and
(h) less than 30% (v/v) of other excipients.

In another aspect, the present invention provides a method of treating a mammal comprising intranasally administering to the mammal an effective amount of a composition as described herein. In one embodiment, the mammal suffers from a blood sugar related disorder, for example, type-2 diabetes. In another embodiment, the mammal suffers from mild cognitive disorder. These and other aspects and features of the present invention are described in more detail below.

### DETAILED DESCRIPTION OF THE INVENTION

The invention provides intranasally deliverable pharmaceutical formulations comprising a hypoglycemic agent for use in the treatment of a variety of disorders, for example, type 2 diabetes, that benefit from administration of a hypoglycemic agent. The intranasal formulations address problems faced when using oral formulations, for example, reducing the time delay before effective amounts of the hypoglycemic agent are present in the systemic circulation and reducing absorption variability due to the presence or absence of food in the gut.

### Hypoglycemic Agents

Compositions of the invention comprise at least one hypoglycemic agent. The term “hypoglycemic agent,” as used herein, includes any substance, naturally or synthetically derived, that is effective in the treatment and/or prevention of blood glucose-related diseases and disorders. Unless specifically defined herein, the terms used in this application shall have their plain and ordinary meaning as understood by those skilled in the art of pharmaceutical sciences.

In one embodiment, the hypoglycemic agent is a nonsulfonylurea hypoglycemic agent. In another embodiment, the hypoglycemic agent is a meglitinide. In still another embodiment, the hypoglycemic agent is a phenylacetic acid benzylamide.

In one embodiment, the hypoglycemic agent is a phenylacetic acid benzylamide of Formula (I):

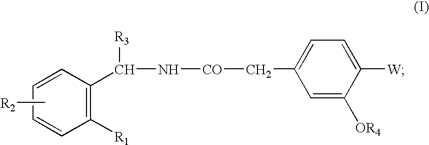

including salts, esters, and prodrugs thereof;
wherein:

R₁ represents an unbranched alkyleneimino group with 4 to 6 carbon atoms optionally mono- or di(allyl of 1 to 3 carbon atoms)-substituted;

R₂ represents hydrogen, halogen, methyl, or methoxy;

R₃ represents a hydrogen atom, an allyl group, an alkyl group with 1 to 7 carbon atoms, a phenyl group optionally substituted by a halogen atom or a methyl or methoxy group, an alkyl group with 1 to 2 carbon atoms substituted by a hydroxy, alkoxy, alkanoyloxy, tetrahydrofuranyl, tetrahydropyranyl, cycloalkyl or phenyl group, in which the alkoxy part can contain from 1 to 3 carbon atoms, the alkanoyloxy part can contain 2 to 3 carbon atoms and the cycloalkyl part can contain 3 to 7 carbon atoms, an alkenyl group with 3 to 6 carbon atoms, an alkynyl group with 3 to 5 carbon atoms, a carboxy group or an alkoxycarbonyl group with a total of 2 to 5 carbon atoms;

R₄ represents a hydrogen atom, a methyl, ethyl, or allyl group; and

W represents a methyl, hydroxymethyl, formyl, carboxyl, alkoxycarbonyl, cyanomethyl, 2-cyanoethyl, 2-cyano-ethenyl, carboxymethyl, 2-carboxyethyl, 2-carboxyethenyl, alkoxycarbonylmethyl, 2-alkoxycarbonyl-ethyl or 2-alkoxycarbonylethenyl group, in which each alkoxy optionally can comprise from 1 to 4 carbon atoms and can be substituted by a phenyl group; and when R₃ is a substistent other than hydrogen and/or the radical R₁ contains an optically active carbon atom, Formula I includes the enantiomers and the diastereomers or their mixtures.

In certain embodiments when W is carboxyl, a non-toxic salt can be formed by the addition of an inorganic or organic base to the carboxyl compound. Alternatively, a non-toxic acid addition salt can be formed by reaction of an inorganic or organic acid with the amino function in the R₁-position.

Specific embodiments of substituents R₁, R₂, R₃, R₄, and W are now discussed in more detail.

R₁ can be pyrrolidino, piperidino, hexamethyleneimino, methyl-pyrrolidino, dimethyl-pyrrolidino, ethyl-pyrrolidino, 2-methyl-piperidino, 3-methyl-piperidino, 4-methyl-piperidino, 3,3-dimethyl-piperidino, cis-3,5-dimethyl-piperidino, trans-3,5-dimethyl-piperidino, ethyl-piperidino, diethyl-piperidino, methyl-ethyl-piperidino, propyl-piperidino, methyl-propyl-piperidino or isopropyl-piperidino.

R₂ can be hydrogen, fluorine, chlorine, bromine, methyl or methoxy.

R₃ can be hydrogen, methyl, ethyl, n-propyl, isopropyl, n-butyl, isobutyl, sec-butyl, tert-butyl, n-pentyl, 2-methyl-n-butyl, 3-methyl-n-butyl, 2,2-dimethylpropyl-n-hexyl, 4-methyl-n-pentyl, n-heptyl, phenyl, fluorophenyl, chlorophenyl, bromophenyl, methylphenyl, methoxyphenol, 1-propen-1-yl, 2-methyl-1-propen-1-yl, 3-methyl-3-buten-2-yl, 2-propen-1-yl, 2-methyl-2-propen-1-yl, 2-buten-1-yl, 2-methyl-2-buten-1-yl, 3-methyl-2-buten-1-yl, 2-buten-1-yl, 2-methyl-3-buten-1-yl, 3-methyl-3-buten-1-yl, 2-hexen-1-yl, 1-propyn-1-yl, 2-propyn-1-yl, 2-butyn-1-yl, 2-pentyn-1-yl, hydroxymethyl, 1-hydroxy-ethyl, 2-hydroxy-ethyl, methoxymethyl, ethoxymethyl, n-propoxymethyl, isopropoxymethyl, 1-methoxy-ethyl, 2-methoxyethyl, 1-ethoxy-ethyl, 2-ethoxy-ethyl, 2-n-propoxy-ethyl, 2-isopropoxy-ethyl, acetoxymethyl, propionyloxymethyl, 1-acetoxy-ethyl, 2-acetoxy-ethyl, 1-propionyloxy-ethyl, 2-propionyloxy ethyl, tetrahydrofuran-2-yl-methyl, 2-(tetrahydrofuran-2-yl)-ethyl, tetrahydrofuran-3-yl-methyl, tetrahydropyran-2-yl-methyl, 2-(tetrahydropyran-2-yl)-ethyl, tetrahydropyran-3-yl-methyl, cyclopropyl-methyl, cyclobutyl-methyl, cyclopentylmethyl), cyclohexylmethyl, cycloheptylmethyl, 2-cyclopropyl-ethyl, 2-cyclobutyl-ethyl, 2-cyclopenyl-ethyl, 2-cyclohexyl-ethyl, 2-cycloheptyl-ethyl, benzyl, 1-phenyl-ethyl, 2-phenyl-ethyl, carboxy, methoxycarbonyl, ethoxycarbonyl, n-propoxycarbonyl, isopropoxycarbonyl, n-butoxycarbonyl, sec-butoxycarbonyl, isobutoxycarbonyl or tert-butoxycarbonyl.

R₄ can be hydrogen, methyl, ethyl, n-propyl, isopropyl, or allyl.

W can be methyl, hydroxymethyl, formyl, carboxy, methoxycarbonyl, ethoxycarbonyl, n-propoxycarbonyl, isopropoxycarbonyl, n-butoxycarbonyl, sec-butoxycarbonyl, isobutoxycarbonyl, tert-butoxycarbonyl, benzyloxycarbonyl, 1-phenylethoxycarbonyl, 2-phenylethoxycarbonyl, 3-phenylpropoxycarbonyl, cyanomethyl, 2-cyano-ethyl, 2-cyano-ethenyl, carboxy-methyl, methoxycarbonylmethyl, ethoxycarbonyl-methyl, n-propoxycarbonylmethyl, n-butoxycarbonyl-methyl, tert-butoxycarbonylmethyl, 2-methoxycarbonyl-ethyl, 2-ethoxycarbonyl-ethyl, 2-n-propoxycarbonyl-ethyl, 2-isopropoxycarbonyl-ethyl, 2-n-butoxycarbonyl-ethyl, 2-tert-butoxycarbonyl-ethyl, 2-methoxycarbonyl-ethenyl, 2-ethoxycarbonyl-ethenyl, 2-n-propoxy-ethenyl or 2-tert-butoxycarbonylethenyl.

In another embodiment, suitable hypoglycemic agents are compounds of Formula I wherein:

R₁ represents a pyrrolidino, piperidino, 4-methyl-piperidino, 3-methylpiperidino, 3,3-dimethyl-piperidino, 3,5-dimethyl-piperidino or hexamethyleneimino group;

R₂ is a hydrogen, fluorine or chlorine atom;

R₃ is a hydrogen atom, an alkyl group with 1 to 6 carbon atoms, a phenyl, methyl-phenyl, chloro-phenyl, methoxy-phenyl, cyclopropylmethyl, cyclobutylmethyl, cyclopentylmethyl, cyclohexylmethyl, tetrahydrofuran-2-yl-methyl, tetrahydropyran-2-yl-methyl, propargyl, hydroxymethyl, ethoxymethyl, acetoxymethyl, propionyloxymethyl, carboxy, methoxycarbonyl, ethoxycarbonyl or propoxycarbonyl group or a branched or unbranched alkenyl group with 3 or 4 carbon atoms;

R₄ is a methyl, ethyl or allyl group;

W is a methyl, hydroxymethyl, formyl, carboxyl, benzyloxycarbonyl, carboxymethyl, methoxycarbonylmethyl, ethoxycarbonylmethyl, cyanomethyl, 2-carboxy-ethyl, 2-ethoxycarbonyl-ethyl, 2-cyano-ethyl, 2-carboxy-ethenyl, 2-ethoxycarbonyl-ethenyl or 2-cyano-ethenyl group or an alkoxycarbonyl group with 1 to 4 carbon atoms in the alkoxy part; provided that when R₃ is other than hydrogen and/or R₁ represents the 3-methylpiperidino group, Formula I includes the enantiomers and the diastereomers or their mixtures.

In certain embodiments when W is carboxyl, a non-toxic salt can be formed by the addition of an inorganic or organic base to the carboxyl compound. Alternatively, a non-toxic acid addition salt can be formed by reaction of an inorganic or organic acid with the amino function in the R₁-position.

In another embodiment, suitable hypoglycemic agents are compounds of Formula I wherein: R₁ represents a piperidino group; R₂ represents a hydrogen atom; R₃ represents an alkyl group with 1 to 6 carbon atoms, an alkenyl group with 3 to 4 carbon atoms, a phenyl, tetrahydropyran-2-yl-methyl, cyclopropylmethyl or cyclohexylmethyl group; R₄ represents a methyl ethyl or allyl group; and W represents a carboxyl, methoxycarbonyl, ethoxycarbonyl or cyanomethyl group.

In certain embodiments when W is carboxyl, a non-toxic salt can be formed by the addition of an inorganic or organic base to the carboxyl compound. Alternatively, a non-toxic acid addition salt can be formed by reaction of an inorganic or organic acid with the piperidino moiety.

In another embodiment, suitable hypoglycemic agents are those compounds of Formula I wherein R₁ represents a piperidino group; R₂ represents a hydrogen atom; R₃ represents an alkyl group with 3 to 6 carbon atoms, an alkenyl group with 3 to 4 carbon atoms, a phenyl, cyclopropylmethyl or cyclohexylmethyl group; R₄ represents a methyl or ethyl group; and W represents a carboxyl group. In another embodiment, suitable hypoglycemic agents are those compounds of Formula I, wherein R₃ represents an alkyl group with 3 to 6 carbon atoms, a 2-methyl-1-propen-1-yl, cyclomethylpropyl or cyclohexylmethyl group.

In certain embodiments, when W is carboxyl, a non-toxic salt can be formed by the addition of an inorganic or organic base to the carboxyl compound. Alternatively, a non-toxic acid addition salt can be formed by reaction of an inorganic or organic acid with the with the piperidino moiety.

In yet another embodiment, suitable hypoglycemic agents are those compounds of the Formula I wherein R₃ represents a n-propyl, n-butyl, isobutyl, sec-butyl, n-pentyl, 2-methyl-1-propen-1-yl, cyclomethylpropyl or cyclohexylmethyl group.

In another embodiment, the hypoglycemic agent is 2-ethoxy-4-[N-(1-(2-piperidino-phenyl)-1-butyl)-aminocarbonyl methyl]-benzoic acid.

In another embodiment, the hypoglycemic agent is 2-ethoxy-4-[N-(1-(2-piperidino-phenyl)-3-methyl-1-butyl)-aminocarbonylmethyl]-benzoic acid.

In another embodiment, the hypoglycemic agent is form (A) of 2-ethoxy-4-[N-(1-(2-piperidino-phenyl)-3-methyl-1-butyl)-aminocarbonylmethyl]-benzoic acid, recrystallized from acetone/petroleum ether, having a melting point of 90 to 92° C. In another embodiment, the hypoglycemic agent is form (B) of 2-ethoxy-4-[N-(1-(2-piperidino-phenyl)-3-methyl-1-butyl)-aminocarbonylmethyl]-benzoic acid, recrystallized from ethanol/water, having a melting point of 140 to 142° C. In another embodiment, the hypoglycemic agent is form (C) of 2-ethoxy-4-[N-(1-(2-piperidino-phenyl)-3-methyl-1-butyl)-aminocarbonylmethyl]-benzoic acid, recrystallized from methanol, having a melting point of 74 to 85° C. In another embodiment, the hypoglycemic agent is 2-ethoxy-4-[N-(alpha-cyclohexylmethyl-2-piperidino-benzyl)-aminocarbonylmethyl]-benzoic acid; the enantiomers thereof or their mixtures; a non-toxic salt thereof formed with an inorganic or organic base; or a non-toxic acid addition salt formed by an inorganic or organic acid with the piperidino moiety.

In another embodiment, the hypoglycemic agent composition comprises a compound of Formula II:

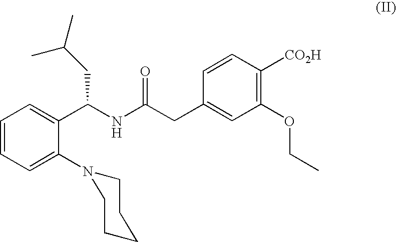

and salts, esters, and prodrugs thereof.

It is understood that compositions of the invention can comprise one or more hypoglycemic agents in any suitable amount. In one embodiment, a composition of the invention comprises a hypoglycemic agent in an amount of about 1 μg to about 1000 mg, about 1 μg to about 500 mg, about 1 g to about 250 mg or about 1 μg to about 200 mg. Compositions of the invention typically comprise one or more hypoglycemic agents in a concentration of about 0.1 mg/mL to about 300 mg/mL, about 0.5 mg/mL to about 250 mg/mL, about 0.75 mg/mL to about 200 mg/mL, or about 1 mg/mL to about 100 mg/mL.

In another embodiment, where the hypoglycemic agent is repaglinide or a salt, ester, or enantiomer thereof, a composition of the invention comprises from about 0.01 to about 20 mg, from about 0.1 to about 15 mg, or from about 0.25 to about 10 mg of repaglinide, for example about 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.5, 4.7, 4.8, 4.9 or 5 mg.

Suitable hypoglycemic agents, and methods for making the same, are disclosed in U.S. Pat. Nos. 5,312,924; 6,143,769; 6,677,358; RE 37,035; 5,488,150; 6,559,188; 6,641,841; 6,844,008; 6,878,749; and RE 34,878.

### Liquid Nasal Carrier

The hypoglycemic agent is formulated with a liquid nasal carrier. As used herein, the term “liquid nasal carrier” or “liquid carrier” refers to a liquid vehicle (e.g., solution, emulsion, or suspension) designed for delivery of a drug to the nasal mucosa of a subject. The liquid nasal carrier can include one or more excipients such as diluents, solvents and/or co-solvents suitable for application to the nasal mucosa. Suitable diluents include aqueous or non-aqueous diluents or combination thereof. Examples of aqueous diluents include, but are not limited to, saline, water, water for injection (WFI), dextrose or combinations thereof.

In one embodiment, the liquid nasal carrier comprises a solvent such as a water miscible solvent. Non-limiting examples of suitable solvents include alcohol, for example, ethanol, and isopropylalcohol, buffers, for example, a phosphate buffer, propylene glycol, glycerol, polyethylene glycol, for example, PEG-200, PEG-300, PEG-350, PEG-400, or PEG-450, tetra (ethylene glycol), and methoxy-polyethylene glycol.

Any desired aqueous and/or non-aqueous diluents, solvents or co-solvents can be added in various concentrations and combinations to form a liquid nasal carrier in compositions of the invention. The liquid nasal carrier can be present in any suitable amount, for example about 10% to about 99%, about 20% to about 98%, about 30% to about 97%, by weight of the composition. In another embodiment, the liquid nasal carrier can be added to the other components of the composition in an amount sufficient to q.s. the composition to a desired final volume. In one embodiment, at least a portion of, at least about 20% of, at least about 30%, at least about 40%, at least about 50%, at least about 60%, at least about 70%, at least about 80%, or at least about 90%, by weight, of the hypoglycemic agent is in dissolved and/or solubilized form in the liquid nasal carrier.

### Pharmaceutical Excipients

Compositions of the invention optionally comprise one or more additional pharmaceutically acceptable excipients. The term “excipient,” as used herein is understood to mean any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Illustrative excipients include antioxidants, surfactants, adhesives, agents to adjust the pH and osmolarity, preservatives, thickening agents, sweetening agents, flavoring agents, taste masking agents, colorants, buffering agents, and penetration enhancers. Generally speaking, a given excipient, if present, will be present in an amount of about 0.001% to about 95%, about 0.01% to about 80%, about 0.02% to about 25%, or about 0.3% to about 10%, by weight.

Illustrative antioxidants for use in the present invention include, but are not limited to, butylated hydroxytoluene, butylated hydroxyanisole, potassium metabisulfite, vitamin E, tertiary-butyl hydroquinone, and the like. One or more antioxidants, if desired, are typically present in a composition of the invention in an amount of about 0.01% to about 2.5%, for example about 0.01%, about 0.05%, about 0.1%, about 0.5%, about 1%, about 1.5%, about 1.75%, about 2%, about 2.25%, or about 2.5%, by weight.

In various embodiments, compositions of the invention comprise a preservative. Ideally, the optional preservative will be present in quantities sufficient to preserve the composition, but in quantities low enough that they do not cause irritation of the nasal mucosa. Suitable preservatives include, but are not limited to, benzalkonium chloride; methyl-, ethyl-, propyl- or butyl-paraben; benzyl alcohol; phenylethyl alcohol; benzethonium; or a combination thereof. Typically, the optional preservative is present in an amount of about 0.01% to about 0.5% or about 0.01% to about 2.5%, by weight.

In other embodiments, compositions of the invention are preservative-free. As used herein, the term “preservative-free” includes compositions that do not contain any preservative. Thus, in various embodiments, the composition does not contain, for example, benzalkonium chloride; methyl-, ethyl-, propyl- or butyl-paraben; benzyl alcohol; phenylethyl alcohol; or benzethonium.

In one embodiment, compositions of the invention optionally comprise a buffering agent. The optional buffering agent, if present, is present in a composition of the invention in an amount that does not irritate the nasal mucosa. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein. One or more buffering agents, if desired, are present in compositions of the invention in an amount of about 0.01% to about 5% or about 0.01% to about 3%, by weight.

In one embodiment, compositions of the invention optionally comprise one or more surfactants. Optional surfactants typically are present in a composition of the invention in an amount of from about 0.1 mg/mL to about 10 mg/mL, from about 0.5 mg/mL to 5 mg/mL or about 1 mg/mL.

In various embodiments, compositions of the invention may include one or more agents that increase viscosity. Illustrative agents that increase viscosity include, but are not limited to, methylcellulose, carboxymethylcellulose sodium, ethylcellulose, carrageenan, carbopol, and/or combinations thereof. Typically, one or more viscosity increasing agents, if desired, are present in compositions of the invention in an amount of about 0.1% to about 10%, or about 0.1% to about 5%, by weight.

In various embodiments, compositions of the invention comprise one or more sweeteners and/or flavoring agents. Suitable sweeteners and/or flavoring agents include any agent that sweetens or provides flavor to a pharmaceutical composition. The sweetener or flavoring agent will help mask any bitter or bad taste that may occur if the pharmaceutical composition drips back into the mouth after intranasal administration. By addition of a sweetener or flavoring agent to the intranasal composition, a barrier that a patient may have to taking the intranasal composition because of unpleasant taste can be reduced. Optional sweetening agents and/or flavoring agents are typically present in a composition of the invention in an amount of from about 0.1 mg/mL to about 10 mg/mL, from about 0.5 mg/mL to 5 mg/mL or about 1 mg/mL.

Illustrative sweeteners or flavoring agents include, without limitation, acacia syrup, anethole, anise oil, aromatic elixir, benzaldehyde, benzaldehyde elixir, cyclodextrins, compound, caraway, caraway oil, cardamom oil, cardamom seed, cardamom spirit, compound, cardamom tincture, compound, cherry juice, cherry syrup, cinnamon, cinnamon oil, cinnamon water, citric acid, citric acid syrup, clove oil, cocoa, cocoa syrup, coriander oil, dextrose, eriodictyon, eriodictyon fluidextract, eriodictyon syrup, aromatic, ethylacetate, ethyl vanillin, fennel oil, ginger, ginger fluidextract, ginger oleoresin, dextrose, glucose, sugar, maltodextrin, glycerin, glycyrrhiza, glycyrrhiza elixir, glycyrrhiza extract, glycyrrhiza extract pure, glycyrrhiza fluidextract, glycyrrhiza syrup, honey, isoalcoholic elixir, lavender oil, lemon oil, lemon tincture, mannitoi, methyl salicylate, nutmeg oil, orange bitter, elixir, orange bitter, oil, orange flower oil, orange flower water, orange oil, orange peel, bitter, orange peel sweet, tincture, orange spirit, compound, orange syrup, peppermint, peppermint oil, peppermint spirit, peppermint water, phenylethyl alcohol, raspberry juice, raspberry syrup, rosemary oil, rose oil, rose water, stronger, saccharin, saccharin calcium, saccharin sodium, sarsaparilla syrup, sarsaparilla compound, sorbitol solution, spearmint, spearmint oil, sucrose, sucralose, syrup, thyme oil, tolu balsam, tolu balsam syrup, vanilla, vanilla tincture, vanillin, wild cherry syrup, or combinations thereof.

Illustrative taste masking agents include, but are not limited to, cyclodextrins, cyclodextrins emulsions, cyclodextrins particles, cyclodextrins complexes, or combinations thereof.

The foregoing excipients can have multiple roles as is known in the art. For example, some flavoring agents can serve as sweeteners as well as a flavoring agent. Therefore, classification of excipients above is not to be construed as limiting in any manner.

Pharmaceutical compositions as disclosed herein are not limited to any particular pH. In one embodiment, the pH of the composition of the invention ranges from 3 to 9, from 3 to 6, or from 4 to 6, for example, about 5. If adjustment of pH is needed, it can be achieved by the addition of an appropriate acid, such as, for example, hydrochloric acid, or base, such as, for example, sodium hydroxide. It is noted repaglinide was often more stable in solutions having a pH of less than or equal to 9.

Pharmaceutical compositions of the invention can be prepared in any suitable manner. In one embodiment, the compositions are prepared by mixing, in any order, a hypoglycemic agent with a liquid nasal carrier and one or more optional excipients at room temperature under aseptic conditions. When the formulation contains an organic solvent, for example, ethanol, the hypoglycemic acid is first dissolved in the organic solvent. Afterwards, the aqueous solvents and excipients are added to the solution of the hypoglycemic acid dissolved in the organic solvent. In other embodiments, the mixture can be prepared under non-aseptic conditions and then filter sterilized, autoclaved or otherwise sterilized and packaged in a delivery device. It will be understood by those of ordinary skill in the art that the order of mixing is not critical, and the present invention includes without limitation mixing of compositions of the invention in any order. Compositions resulting from such processes represent further embodiments of the invention.

Certain exemplary formulations comprise, for example, 1-2% (w/v) active ingredient (e.g., repaglinide), 0-10% (v/v) alcohol (e.g., ethanol), 0-10% (v/v) propylene glycol, 30-60% (v/v) polyethylene glycol (e.g., PEG-200, PEG-300 or PEG-400), 0-20% (v/v) methoxypolyethylene glycol, 0-40% (v/v) tetra (ethylene glycol), 0-10% (v/v) phosphate buffer, and 0-30% (v/v) other excipients. Other exemplary formulations comprise, for example, 1-2% (w/v) active ingredient (e.g., repaglinide), 5% (v/v) or less of ethanol, 0-10% (v/v) propylene glycol, 50-60% (v/v) PEG-300, 0-20% (v/v) methoxypolyethylene glycol, 0-40% (v/v) tetra (ethylene glycol), 0-10% (v/v) phosphate buffer, and 0-30% (v/v) of other excipients.

### Stability

In one embodiment, a composition of the invention comprises at least 85%, at least 87%, at least 90%, at least 92%, at least 95%, at least 97%, or at least 99% of the original hypoglycemic agent (e.g., repaglinide) after storage (closed or open vessel) at 40° C. and 75% relative humidity for a period of at least 1 week, at least 2 weeks, at least 3 weeks, at least 4 weeks, at least 6 weeks, at least 8 weeks, at least 10 weeks, at least 15 weeks, at least 20 weeks, at least 25 weeks, at least 30 weeks, at least 35 weeks, at least 40 weeks, at least 45 weeks, or at least 50 weeks The formulations described in Table 1 below have shown good stability for a period of about one month. In particular, the formulations described in Example 1 below have shown no precipitation and have remained clear and colorless for a period of about one month.

### Method of Treatment

Compositions of the invention are useful in treating and/or preventing, inter alia, blood glucose related diseases and disorders, such as, hyperglycemia and diabetes, for example type-1 or type-2 diabetes. The compositions may also be useful in treating and/or preventing mild cognitive disorder. Mild cognitive disorder is an art-recognized term for a disorder generally characterized by a decrease in cognitive ability, e.g., memory. The decrease in cognitive ability is generally greater than that attributable to aging, but not as severe as the decrease is cognitive ability observed in patients suffering from dementia.

In one embodiment, the present invention provides a method for treating and/or preventing any of the above disorders in a subject in need thereof comprising intranasally administering to a subject a therapeutically effective amount of a composition described herein, including the formulations described in Table 1. As used herein, the term “effective amount” is understood to mean an amount of drug or agent that is sufficient to elicit the required or desired therapeutic and/or prophylactic response. The intranasal administration can occur 1 to 30, 1 to 20, 1 to 10 or 1 to 5 times per day, per week, or per month.

In another embodiment, compositions of the invention are administered to a subject in an amount of about 0.0005 to about 2 mg/kg body weight, about 0.001 mg/kg body weight, about 0.001 to about 0.5 mg/kg body weight.

In another embodiment, where the active ingredient is repaglinide, compositions of the invention are administered to a subject in an amount sufficient to provide the subject with 0.1 to 20 mg, 0.1 to 15 mg, 0.1 to 10 mg, or 0.1 to 5 mg of repaglinide per day.

In another embodiment, compositions of the invention are administered to a subject that is concurrently taking metformin. In a related embodiment, a composition of the invention is co-administered with metformin.

In another embodiment, a composition of the invention is administered to a subject during the time when the subject is eating a meal, or about not more than about 15 minutes, not more than about 10 minutes, not more than about 5 minutes or not more than about 1 minute before the subject eats a meal, or not more than about 1, not more than about 5, not more than about 10 or not more than about 15 minutes after the meal, or in cases of sickness, when hyperglycemia is evident.

In one embodiment, where the drug being delivered is repaglinide, upon intranasal administration of a composition of the invention to a subject, the subject exhibits one or more of: a Tₘₐₓ repaglinide plasma concentration of at latest about 0.25 hr, about 0.5 hour, or about 0.75 hr; a Cₘₐₓ repaglinide plasma concentration of at least about 10 ng/mL per mg of repaglinide administered, for example about 10 ng/mL per mg repaglinide to about 40 ng/mL repaglinide; and/or an AUC repaglinide plasma concentration of at least about 75 ng*hr/mL/mg of repaglinide administered, for example about 75 to about 200 ng*hr/mL/mg of repaglinide administered. In a related embodiment, the above PK parameters result after administration of a composition in an amount sufficient to provide the subject with about 0.5 to about 4 mg of repaglinide.

In another embodiment, where the drug being delivered is repaglinide, upon intranasal administration of a composition of the invention to a subject in an amount sufficient to provide about 0.5 mg of repaglinide, the subject exhibits one or more of: a Tₘₐₓ repaglinide plasma concentration of at latest about 0.25 hr, about 0.5 hour, or about 0.75 hr; a Cₘₐₓ repaglinide plasma concentration of at least about 8 ng/ mL, about 10 ng/mL, about 15 ng/mL or about 20 ng/mL, for example about 8 ng/mL to about 30 ng/mL; and/or an AUC repaglinide plasma concentration of at least about 60 ng*hr/mL, at least about 70 ng*hr/mL, or at least about 80 ng*hr/mL for example about 60 to about 200 ng*hr/mL. In a related embodiment, the above PK parameters result after administration of a composition in an amount sufficient to provide the subject with about 0.5 to about 4 mg of repaglinide.

In another embodiment, where the drug being delivered is repaglinide, upon intranasal administration of a composition of the invention to a subject in an amount sufficient to provide about 1 mg of repaglinide, the subject exhibits one or more of: a Tₘₐₓ repaglinide plasma concentration of at latest about 0.25 hr, about 0.5 hour, or about 0.75 hr; a Cₘₐₓ repaglinide plasma concentration of at least about 15 ng/ mL, about 18 ng/mL, about 20 ng/mL or about 25 ng/mL, for example, from about 12 ng/mL to about 30 ng/mL; and/or an AUC repaglinide plasma concentration of at least about 80 ng*hr/mL, at least about 120 ng*hr/mL, or at least about 130 ng*hr/mL, for example, about 80 to about 200 ng*hr/mL.

In another embodiment, where the drug being delivered is repaglinide, upon intranasal administration of a composition of the invention to a subject in an amount sufficient to provide about 2 mg of repaglinide, the subject exhibits one or more of: a Tₘₐₓ repaglinide plasma concentration of at latest about 0.25 hr, about 0.5 hour, or about 0.75 hr; a Cₘₐₓ repaglinide plasma concentration of at least about 15 ng/ mL, about 20 ng/mL, about 25 ng/mL or about 30 ng/mL, for example about 15 ng/mL to about 45 ng/mL; and/or an AUC repaglinide plasma concentration of at least about 80 ng*hr/mL, at least about 120 ng*hr/mL, or at least about 150 ng*hr/ml for example about 80 to about 250 ng*hr/mL.

In another embodiment, where the drug being delivered is repaglinide, upon intranasal administration of a composition of the invention to a subject in an amount sufficient to provide about 4 mg of repaglinide, the subject exhibits one or more of: a Tₘₐₓ repaglinide plasma concentration of at latest about 0.25 hr, about 0.5 hour, or about 0.75 hr; a Cₘₐₓ repaglinide plasma concentration of at least about 30 ng/ mL, about 40 ng/mL, about 50 ng/mL or about 60 ng/mL, for example about 30 ng/mL to about 100 ng/mL; and/or an AUC repaglinide plasma concentration of at least about 200 ng*hr/mL, at least about 250 ng*hr/mL, or at least about 450 ng*hr/mL, for example, from about 200 to about 500 ng*hr/mL.

### Delivery Device

Compositions of the present invention can be administered using any suitable intranasal delivery device. In one embodiment, the delivery device is a unit-dose delivery device. Non-limiting examples of suitable intranasal delivery devices, or components thereof, are disclosed in U.S. Pat. Nos. 4,946,069; 5,307,953; 5,368,201; 5,395,032; 5,427,280; 5,482,193; 5,584,417; 5,813,570; 5,893,484; 5,944,222; 5,964,417; 5,967,369; 6,062,433; 6,257,454; 6,626,379; 6,321,942; 6,367,473; and 6,948,492.

The delivery device can be filled with single or multidose amounts of a hypoglycemic agent as described herein. In one embodiment, the invention provides a vessel or reservoir for holding the pharmaceutical composition. In one embodiment, the parts of the device that are in contact with the pharmaceutical composition can be constructed and assembled in a configuration so as to allow for sterilization. Devices with one or more unit-dose(s) can be sterilized either before or after filling and/or packaging, employing methods and technologies that are well known in the art. Individual devices can be packaged, sterilized and shipped; alternatively, entire shipping and storage packages can be sterilized at once, and the devices removed individually for dispensing, without affecting the sterility of the remaining units.

In one embodiment, the volume of liquid contained in each vessel of a delivery device is from about 0.025 mL to about 2 mL, from about 0.25 mL to 1 mL, or from about 0.05 mL to about 0.15 mL.

In another embodiment, a composition of the invention, upon being discharged from an intranasal spray device at a spray distance of 3 cm from a detection laser, for example at a discharge volume of about 100 μL per spray, exhibits a droplet size distribution having a mean Dv10 of about 5 to about 50 μm, about 7.5 to about 40 μm, or about 10 to about 35 μm; a mean Dv50 of about 15 to about 80 μm, about 20 to about 70 μm, or about 30 to about 60 μm; and/or a mean Dv90 of about 40 to about 130 μm, about 50 to about 120 μm, or about 60 to about 100 μm. In another embodiment, the spray has a mean span [(Dv90-Dv10/Dv50)] of about 1 to about 5, about 1.25 to about 4, or about 1.5 to about 3.

In another embodiment, a composition of the invention, upon being discharged from an intranasal spray device at a spray distance of 3 cm from a detection laser, for example, at a discharge volume of about 100 μL per spray, exhibits a droplet size distribution having a mean Dv10 of less than about 20 μm. In another embodiment, a composition of the invention, upon being discharged from an intranasal spray device, for example at a discharge volume of about 100 μL per spray, exhibits a droplet size distribution having a mean Dv10 of less than about 10 μm as measured by cascade impaction. In another embodiment, a composition of the invention, upon being discharged from an intranasal spray device at a spray distance of 3 cm from a detection laser, for example at a discharge volume of about 100 μL per spray, exhibits a droplet size distribution having a mean Dv50 of from about 20 μm to about 90 μm. In another embodiment, a composition of the invention, upon being discharged from an intranasal spray device at a spray distance of 3 cm from a detection laser, for example at a discharge volume of about 100 μL per spray, exhibits a droplet size distribution having a mean Dv90 of from about 50 μm to about 60 μm.

In a related embodiment, upon positioning the device 3 cm away from an impaction plate, actuating the device to produce a spray pattern onto the impaction plate, and measuring the diameter of the spray pattern, the spray pattern has a maximum diameter (Dₘₐₓ) of about 1 to about 4 cm, about 2 to about 3 cm or about 2.2 to about 2.5 cm, for example about 2.3 cm. In another related embodiment, the spray has a minimum diameter (Dₘᵢₙ) of about 1 to about 3 cm, about 1.5 to about 2.8 cm or about 1.8 to about 2.3 cm, for example about 2.1 cm.

### EXAMPLES

The invention now being generally described, will be more readily understood by reference to the following examples, which are included merely for purposes of illustration of certain aspects and embodiments of the present invention, and are not intended to limit the scope of the invention in any way.

### Example 1

The formulations described in this Example were prepared for intranasal administration using the following reagents, Repaglinide USP obtained from USV Limited, USP/NF grade ethanol [EtOH], 190 proof, obtained from Sigma-Aldrich, propylene glycol (PG), USP/FCC grade, obtained from J.T. Baker, tetra (ethylene glycol) (T-EG), obtained from Aldrich, polyethylene glycol (PEG-300) obtained from Aldrich, polyethylene glycol (PEG-400) obtained from Spectrum, glycerin, EP/USP, obtained from EM Science, methoxypolyethylene glycol (M-PEG) with an average mw of 350 g/mol, obtained from Sigma, benzyl alcohol (BNZ-OH), ACS grade reagent, obtained from Aldrich, potassium phosphate dibasic (K₂HPO₄), USP grade, obtained from EMD, potassium phosphate monobasic (KH₂PO₄), FCC grade, obtained from Mallinckrodt.

Fourteen different formulations created using the foregoing reagents are listed in Table 1.

TABLE 1

| Formula 1 | Formula 2 | Formula 3 |
|---|---|---|
| 10 mg Repaglinide | 10 mg Repaglinide | 10 mg Repaglinide |
| 0.2 mL EtOH | 0.1 mL EtOH | 0.1 mL EtOH |
| 0.5 mL PEG-300 | 0.3 mL PG | 0.1 mL PG |
| 0.3 mL M-PEG | 0.4 mL PEG-300 | 0.5 mL PEG-300 |
| | 0.2 mL M-PEG | 0.3 mL M-PEG |
| Formula 4 | Formula 5 | Formula 6 |
| 10 mg Repaglinide | 10 mg Repaglinide | 10 mg Repaglinide |
| 0.1 mL EtOH | 0.1 mL EtOH | 0.1 mL EtOH |
| 0.6 mL PEG-300 | 0.1 mL PG | 0.6 mL PEG-300 |
| 0.3 mL M-PEG | 0.5 mL PEG-300 | 0.2 mL M-PEG |
| | 0.2 mL M-PEG | 0.1 mL phosphate |
| | 0.1 mL phosphate | buffer pH 8.0 |
| | buffer pH 8.0 | |
| Formula 7 | Formula 8 | Formula 9 |
| 10 mg Repaglinide | 10 mg Repaglinide | 10 mg Repaglinide |
| 0.05 mL EtOH | 0.05 mL EtOH | 0.5 mL PEG-300 |
| 0.5 mL PEG-300 | 0.5 mL PEG-300 | 0.4 mL T-EG |
| 0.35 mL T-EG | 0.35 mL T-EG | 0.1 mL phosphate |
| 0.1 mL phosphate | 0.1 mL phosphate | buffer pH 8.9 |
| buffer pH 8.9 | buffer pH 8.0 | |
| Formula 10 | Formula 11 | Formula 12 |
| 10 mg Repaglinide | 10 mg Repaglinide | 20 mg Repaglinide |
| 0.5 mL PEG-300 | 0.05 mL BNZ-OH | 0.05 mL EtOH |
| 0.4 mL T-EG | 0.5 mL PEG-300 | 0.5 mL PEG-300 |
| 0.1 mL phosphate | 0.25 mL T-EG | 0.35 mL T-EG |
| buffer pH 8.0 | 0.2 mL phosphate | 0.1 mL phosphate |
| | buffer pH 8.0 | buffer pH 8.0 |
| Formula 13 | Formula 14 | |
| 20 mg Repaglinide | 10 mg Repaglinide | |
| 0.05 mL EtOH | 0.1 mL EtOH | |
| 0.5 mL PEG-300 | 0.4 mL T-EG | |
| 0.45 mL T-EG | 0.4 mL PEG-300 | |
| | 0.1 mL phosphate | |
| | buffer pH 8.0 | |
| | 0.005 mL peppermint | |
| | oil | |

The formulations described in Table 1, when tested, were found to provide superior solubility and stability for repaglinide. This observation was surprising because repaglinide was found to be poorly soluble or insoluble in 100% methoxy-polyethylene glycol, 350. For example, in one formulation made from 100% methoxy-polyethylene glycol, 350, the repaglinide was not completely solubilized, and the formulation had a yellow color indicating degradation. The repaglinide also precipitated from the solution upon storage. Although the administration of a reference formulation consisting of 100% methoxy-polyethylene glycol and repaglinide (concentration of approximately 2 mg/mL repaglinide) to an adult male subject with type-2 diabetes caused a decrease in the subject's blood glucose level, this formulation was extremely irritating to the nasal passage.

### Example 2

It is contemplated that each of the formulations set forth in Example 1 can be administered intranasally to a patient suffering from type-2 diabetes to ameliorate one or more symptoms of type-2 diabetes. The dosage and dosing schedule can be determined by one of ordinary skill in the art using standard procedures.

By way of example, formulations 1 and 2 from Example 1 were administered intranasally using a unit dose spray device to a human male subject having type-2 diabetes. Formula 1 and Formula 2 were administered to each nariz of the subject within 2 minutes of each other in order to test for irritancy and absorption. Upon administration, both formulations were significantly less irritating than the reference formulation of Example 1. Furthermore, consecutive administration of Formulae 1 and 2 resulted in the lowering of the blood glucose level from 137 mg/dL to 82 mg/mL, as measured by an Accu-Chek Aviva blood glucose monitor, within 49 minutes following administration.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respect illustrative rather than limiting the invention described herein. The scope of the invention is thus indicated by the appended claims rather than by the foregoing description, and all changes that come within the meaning and range of equivalency of the claims are intended to be embraced therein.

What is claimed is:

1. An intranasally deliverable pharmaceutical composition comprising a therapeutically effective amount of a hypoglycemic agent or pharmaceutically acceptable salt thereof and a liquid nasal carrier, wherein the hypoglycemic agent or salt thereof is dissolved or solubilized in the liquid nasal carrier.

2. The composition of claim 1, wherein the hypoglycemic agent comprises a compound of Formula I 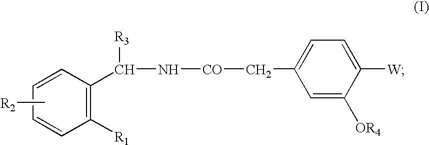
including salts, esters, and prodrugs thereof, wherein:
R₁ represents an unbranched alkyleneimino group with 4 to 6 carbon atoms optionally mono- or di-substituted;
R₂ represents hydrogen, halogen, methyl, or methoxy;
R₃ represents a hydrogen atom, an allyl group, an alkyl group with 1 to 7 carbon atoms, a phenyl group optionally substituted by a halogen atom or a methyl or methoxy group, an alkyl group with 1 to 2 carbon atoms substituted by a hydroxy, alkoxy, alkanoyloxy, tetrahydrofuranyl, tetrahydropyranyl, cycloalkyl or phenyl group, in which the alkoxy part can contain from 1 to 3 carbon atoms, the alkanoyloxy part can contain 2 to 3 carbon atoms and the cycloalkyl part can contain 3 to 7 carbon atoms, an alkenyl group with 3 to 6 carbon atoms, an alkynyl group with 3 to 5 carbon atoms, a carboxy group or an alkoxycarbonyl group with a total of 2 to 5 carbon atoms;
R₄ represents hydrogen, methyl, ethyl or allyl; and
W represents methyl, hydroxymethyl, formyl, carboxyl, alkoxycarbonyl, cyanomethyl, 2-cyanoethyl, 2-cyano-ethenyl, carboxymethyl, 2-carboxyethyl, 2-carboxyethenyl, alkoxycarbonylmethyl, 2-alkoxycarbonyl-ethyl or 2-alkoxycarbonylethenyl, in which each alkoxy optionally contains from 1 to 4 carbon atoms and can be substituted by a phenyl group; and when R₃ is a substituent other than a hydrogen and/or R₁ contains an optically active carbon atom, Formula I includes the enantiomers and the diastereomers thereof or their mixtures.

3. The composition of claim 2, wherein the liquid nasal carrier comprises water.

4. The composition of claim 3, wherein the liquid nasal carrier further comprises at least one pharmaceutically acceptable solvent or co-solvent.

5. The composition of claim 2, wherein R₁ is pyrrolidino, piperidino, hexamethyleneimino, methyl-pyrrolidino, dimethyl-pyrrolidino, ethyl-pyrrolidino, 2-methyl-piperidino, 3-methyl-piperidino, 4-methylpiperidino, 3,3-dimethyl-piperidino, cis-3,5-dimethyl-piperidino, trans-3,5-diimethyl-piperidino, ethyl-piperidino, diethyl-piperidiino, methyl-ethylpiperidino, propyl-piperidino, methyl-propyl-piperidino or isopropylpiperidino.

6. The composition of claim 5, wherein R₂ is hydrogen, fluorine, chlorine, bromine, methyl or methoxy.

7. The composition of claim 6, wherein R₃ is hydrogen, methyl, ethyl, n-propyl, isopropyl, n-butyl, isobutyl, sec-butyl, tert-butyl, n-pentyl, 2-methyl-n-butyl, 3-methyl-n-butyl, 2,2-dimethyl-propyl-n-hexyl, 4-methyl-n-pentyl, n-heptyl, phenyl, fluorophenyl, chlorophenyl, bromophenyl, methylphenyl, methoxyphenol, 1-propen-1-yl, 2-methyl-1-propen-1-yl, 3 -methyl-3-buten-2-yl, 2-propen-1-yl, 2-methyl-2-propen-1-yl, 2-buten-1-yl, 2-methyl-2-buten-1-yl, 3-methyl-2-buten-1-yl, 2-buten-1-yl, 2-methyl-3-buten-1-yl, 3-methyl-3-buten-1-yl, 2-hexen-1-yl, 1-propyn-1-yl, 2-propyn-1-yl, 2-butyn-1-yl, 2-pentyn-1-yl, hydroxymethyl, 1-hydroxy-ethyl, 2-hydroxy-ethyl, methoxymethyl, ethoxymethyl, n-propoxymethyl, isopropoxymethyl, 1-methoxy-ethyl, 2-methoxy-ethyl, 1-ethoxy-ethyl, 2-ethoxy-ethyl, 2-n-propoxy-ethyl, 2-isopropoxy-ethyl, acetoxymethyl, propionyloxymethyl, 1-acetoxy-ethyl, 2-acetoxy-ethyl, 1-propionyloxy-ethyl, 2-propionyloxy ethyl, tetrahydrofuran-2-yl-methyl, 2- (tetrahydrofuran-2-yl)-ethyl, tetrahydrofuran-3-yl-methyl, tetrahydropyran-2-yl-methyl, 2-(tetrahydropyran-2-yl)-ethyl, tetrahydropyran-3-yl-methyl, cyclopropyl-methyl, cyclobutyl-methyl, cyclopentylmethyl), cyclohexylmethyl, cycloheptylmethyl, 2-cyclopropylethyl, 2-cyclobutylethyl, 2-cyclopenyl-ethyl, 2-cyclohexyl-ethyl, 2-cycloheptyl-ethyl, benzyl, 1-phenyl-ethyl, 2-phenyl-ethyl, carboxy, methoxycarbonyl, ethoxycarbonyl, n-propoxycarbonyl isopropoxycarbonyl, n-butoxycarbonyl, sec-butoxycarbonyl, isobutoxycarbonyl or tert-butoxycarbonyl.

8. The composition of claim 7, wherein R₄ is hydrogen, methyl, ethyl, n-propyl, isopropyl, or allyl.

9. The composition of claim 8, wherein W is methyl, hydroxymethyl, formyl, carbonyl, carboxy, methoxycarbonyl, ethoxycarbonyl, n-propoxycarbonyl, isopropoxycarbonyl, n-butoxycarbonyl, sec-butoxycarbonyl, isobutoxycarbonyl, tert-butoxycarbonyl, benzyloxycarbonyl, 1-phenylethoxycarbonyl, 2-phenylethoxycarbonyl, 3-phenylpropoxycarbonyl, cyanomethyl, 2-cyanoethyl, 2-cyano-ethenyl, carboxy-methyl, methoxycarbonylmethyl, ethoxycarbonyl-methyl, n-propoxycarbonylmethyl, n-butoxycarbonylmethyl, tert-butoxycarbonylmethyl, 2-methoxycar-bonyl-ethyl, 2-ethoxycarbonyl-ethyl, 2-n-propoxycarbonyl-ethyl, 2-isopropoxycarbonyl-ethyl, 2-n-butoxycarbonyl-ethyl, 2-tert-butoxycarbonyl-ethyl, 2-methoxycarbonyl-ethenyl, 2-ethoxycarbonyl-ethenyl, 2-n-propoxy-ethenyl or 2-tert-butoxycarbonylethenyl.

10. The composition of claim 2, wherein
R₁ is a piperidino group;
R₂ is a hydrogen atom;
R₃ is selected from the group consisting of an alkyl group with 1 to 6 carbon atoms, an alkenyl group with 3 to 4 carbon atoms, a phenyl, tetrahydropyran-2-yl-methyl, cyclopropylmethyl and cyclohexylmethyl group;
R₄ is selected from the group consisting of methyl, ethyl, and allyl; and
W is selected from the group consisting of carboxyl, methoxycarbonyl, ethoxycarbonyl and cyanomethyl group.

11. The composition of claim 1, wherein the hypoglycemic agent is 2-ethoxy-4-[N-(1-(2-piperidino-phenyl)-1-butyl)-aminocarbonyl methyl]-benzoic acid.

12. The composition of claim 1, wherein the hypoglycemic agent is 2-ethoxy-4-[N-(1-(2-piperidino-phenyl)-3-methyl-1-butyl)-aminocarbonylmethyl]-benzoic acid.

13. The composition of claim 1, wherein the hypoglycemic agent is form (A) of 2-ethoxy-4-[N-(1-(2-piperidino-phenyl)-3-methyl-1-butyl)-aminocarbonylmethyl]-benzoic acid, recrystallized from acetone/petroleum ether, having a melting point of 90 to 92° C.

14. The composition of claim 1, wherein the hypoglycemic agent is form (B) of 2-ethoxy-4-[N-(1-(2-piperidino-phenyl)-3-methyl-1-butyl)-aminocarbonylmethyl]-benzoic acid, recrystallized from ethanol/water, having a melting point of 140 to 142° C.

15. The composition of claim 1, wherein the hypoglycemic agent is form (C) of 2-ethoxy-4-[N-(1-(2-piperidino-phenyl)-3-methyl-1-butyl)-amninocarbonylmethyl]-benzoic acid, recrystallized from methanol, having a melting point of 74 to 85° C.

16. The composition of claim 1, wherein the hypoglycemic agent is 2-ethoxy-4-[N-(alpha-cyclohexylmethyl-2-piperidino-benzyl)-aminocarbonylmethyl]-benzoic acid; the enantiomers thereof or their mixtures; a non-toxic salt thereof formed with an inorganic or organic base; or a non-toxic acid addition salt formed by an inorganic or organic acid with the piperidino moiety.

17. The composition of claim 1, wherein the hypoglycemic agent composition comprises a compound of Formula II: 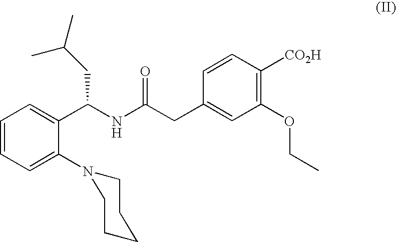
including salts, esters, and prodrugs thereof.

18. The composition of claim 4, wherein the at least one solvent or co-solvent is selected from the group consisting of glycerol, propylene glycol, alcohol, isopropylalcohol, polyethylene glycol, methoxypolyethylene glycol, tetraethylene glycol, and combinations thereof.

19. The composition of claim 1, wherein the composition comprises from about 50% to about 60% (v/v) of a polyethylene glycol having a weight average molecular weight from about 200 g/mol to about 400 g/mol.

20. The composition of claim 1, wherein the composition comprises polyethylene glycol 300.

21. The composition of claim 1, wherein the composition comprises methoxy-polyethylene glycol.

22. The composition of claim 21, wherein the composition comprises from 0 to 20% (v/v) methoxy-polyethylene glycol.

23. The composition of claim 1, wherein the composition comprises tetra (ethylene glycol).

24. The composition of claim 1, wherein the composition comprises:
(a) from 0.5% to 5% (w/v) repaglinide;
(b) from 0% to 10% (v/v) ethanol;
(c) from 0% to 10% (v/v) propylene glycol;
(d) from 30% to 60% (v/v) polyethylene glycol 300;
(e) from 0% to 20% (v/v) methoxy-polyethylene glycol;
(f) from 0% to 40% (v/v) tetra (ethylene glycol);
(g) from 0% to 10% (v/v) phosphate buffer; and
(h) less than 30% (v/v) of other excipients.

25. An intranasally deliverable pharmaceutical composition comprising a formulation set forth in Table 1.

26. A method of treating a disorder treatable with a hypoglycemic agent, the method comprising intranasally administering to a subject in need thereof an effective amount of a composition of claim 1.

27. The method of claim 26, wherein the disorder is type-2 diabetes.

28. The method of claim 26 wherein the disorder is mild cognitive disorder.
